(19) United States
(12) Patent Application Publication
(10) Pub. No.: US 2025/0339624 A1
(43) Pub. Date: Nov. 6, 2025

(54) MEDICINE DELIVERY SYSTEM, METHODS OF PROVIDING FEEDBACK RELATED TO USE OF MEDICINE DELIVERY SYSTEM, AND METHODS OF DETECTING USAGES OF MEDICINE DELIVERY SYSTEM

(71) Applicant: BIGFOOT BIOMEDICAL, INC., Milpitas, CA (US)

(72) Inventors: Michael J. WAKIM, Milpitas, CA (US); Rachael Elizabeth PRICE, Milpitas, CA (US); Olga SMELYANETS, Milpitas, CA (US)

(21) Appl. No.: 18/880,067

(22) PCT Filed: Jun. 29, 2023
(86) PCT No.: PCT/US2023/069452

§ 371 Date: Dec. 30, 2024

Related U.S. Application Data
(60) Provisional application No. 63/367,443, filed on Jun. 30, 2022.

(51) Int. Cl. A61M 5/32; A61M 5/48; G16H 20/17

(52) U.S. Cl. CPC A61M 5/3202; A61M 5/48; G16H 20/17; A61M 2205/332

(57) ABSTRACT

A medicine delivery device includes an injection pen and a pen cap. The pen cap includes one or more detectors, a vibration motor, and a controller. The controller is configured to receive information from the one or more detectors of the pen cap regarding physical interactions between the pen cap and the injection pen, analyze the received information to identify at least one of an incorrect physical interaction or a correct physical interaction between the pen cap and the injection pen, and responsive to identifying the at least one of an incorrect physical interaction or a correct physical interaction between the pen cap and the injection pen, provide feedback via the vibration motor of the medicine delivery device.

### PRIORITY CLAIM

This application claims the benefit of the filing date of U.S. Provisional Patent Application Ser. No. 63/367,443, filed Jun. 30, 2022, for “MEDICINE DELIVERY SYSTEM, METHODS OF PROVIDING FEEDBACK RELATED TO USE OF MEDICINE DELIVERY SYSTEM, AND METHODS OF DETECTING USAGES OF MEDICINE DELIVERY SYSTEM,” the disclosure of which is hereby incorporated herein in its entirety by this reference.

### TECHNICAL FIELD

This disclosure relates generally to medicine delivery systems and methods of improving medication therapy management by providing confirmation for correct usages of a medicine delivery device and generating awareness for incorrect usages of the medicine delivery device.

### BACKGROUND

Drug delivery dosing pens are typically utilized for delivering various medications for numerous different medication therapies. The therapies often include dosing regimens of one or more of growth hormones, insulin, fertility medication, Homozygous Familial Hypercholesterolemia (HoFH) treatment, etc.

In regard to insulin therapy, diabetes mellitus is a chronic metabolic disorder caused by the inability of a person's pancreas to produce sufficient amounts of the hormone insulin such that the person's metabolism is unable to provide for the proper absorption of sugar and starch. This can lead to hyperglycemia or hypoglycemia. Hyperglycemia refers to the presence of an excessive amount of glucose within the blood plasma. Persistent hyperglycemia has been associated with a variety of serious symptoms and life-threatening long-term complications such as dehydration, ketoacidosis, diabetic coma, cardiovascular diseases, chronic renal failure, retinal damage, and nerve damage with the risk of amputation of extremities. Hypoglycemia is the condition where glucose level is lower than the standard range. Hypoglycemia has been associated with a variety of symptoms such as clumsiness, trouble talking, confusion and life-threatening long-term complications such as loss of consciousness, seizures, or death. Because healing is not yet possible, a permanent therapy is necessary which provides constant glycemic control in order to constantly maintain the level of blood glucose within normal limits. Such glycemic control is achieved by regularly supplying external drugs to the body of the patient to thereby reduce the elevated levels of blood glucose.

Often, a permanent therapy is necessary to maintain a proper blood glucose level within normal limits. Maintaining a proper glucose level is achieved by regularly supplying insulin to a person with diabetes (PWD). Maintaining a proper blood glucose level creates a significant cognitive burden for a PWD and affects many aspects of the PWD's life. For example, the cognitive burden on a PWD may be attributed to, among other things, tracking meals and constant check-ins and minor course corrections of blood glucose levels. The adjustments of blood glucose levels by a PWD may include taking insulin, tracking insulin dosing and glucose, deciding how much insulin to take, how often to take it, where to inject the insulin, and how to time insulin doses in relation to meals and/or glucose fluctuations. The foregoing factors make up just a portion of the significant cognitive burden of a PWD.

The following example of a typical daily routine for a PWD further illustrates the significant cognitive burden of a PWD. In the morning, the first thoughts/actions by a PWD are often related to their glucose, such as, what is their blood glucose level? How was their blood glucose level overnight? And how are they currently feeling? Upon checking their blood glucose levels (e.g., using a blood glucose meter or monitor), a PWD may then consider what actions to take, such as adjusting their morning activities, changing when or what to eat for breakfast, or determining to take rapid-acting (RA) insulin and deciding where to inject the rapid-acting (RA) insulin. Before they even eat breakfast (or any meal), a PWD considers the amount of food and types of food they plan to eat, perhaps modifying their RA insulin dose based on the carbohydrate content of the food they choose to eat. Before they administer RA insulin, the PWD will try to remember when they took their last dose of insulin, what happened the last time they ate a particular meal and how they felt.

Before leaving the house, a PWD considers, among other things, whether they have enough supplies for glucose monitoring or insulin dosing. This may include batteries, charged devices, backup supplies, glucose testing supplies, and insulin supplies to treat for high blood glucose levels. Additionally, a PWD needs to consider any physical activities (e.g., walking kids to school, going to the gym, riding a bike) that will affect their glucose because exercise may cause their blood glucose to go lower than expected. Even before driving a vehicle, a PWD checks their glucose to determine if it is at a safe level for driving.

As lunchtime approaches, a PWD considers their glucose prior to eating lunch, such as what time they may expect to eat, what they expect to eat throughout the day. As such, a PWD tallies up the carbohydrates and adjusts insulin doses in their head. A PWD also considers what insulin doses were recently taken and whether those doses may still be working to lower blood glucose. This is all done in parallel with whatever they are doing in their busy day, and so the PWD often forgets or fails to fully consider all of the factors described above.

Throughout the day, a PWD often checks glucose levels, especially on days when their activities vary from a typical day. This constant thinking, checking, planning may be exhausting, especially when each check requires decisions, math, and possible behavior changes. Additionally, during the day, a PWD may check inventory on supplies, speak with a health care provider (HCP), refill prescriptions, and contact their health insurance to discuss their therapy and/or supplies.

In the evening, a PWD may have to take a daily insulin dose of long-acting (LA) insulin. Additionally, the PWD may determine if their glucose is holding steady before they fall asleep. If they use an infusion pump, they have to check if their insulin pump is low on insulin and whether they need to refill it before sleep. If they have a continuous glucose monitor, they have to check and see if it is working. Even then, based on what they ate for dinner, the nighttime insulin might not keep their glucose steady. Glucose levels in the night may interfere with sleep as well as add anxiety that could disrupt sleep.

Accordingly, managing diabetes requires significant attention to detail throughout the day. Even with careful planning and self-monitoring, a PWD may skip doses, double dose, or dose the wrong amount and/or type of insulin. Insufficient insulin may result in hyperglycemia, and too much insulin may result in hypoglycemia, which may result in clumsiness, trouble talking, confusion, loss of consciousness, seizures, or death.

In order to assist with self-treatment, some diabetes treatment devices (e.g., blood glucose meters, insulin pumps, without limitation) are equipped with insulin bolus calculators that have the user input an estimate (e.g., numerical estimate) of the quantity of carbohydrates consumed or about to be consumed (or additionally or alternatively protein, fat, or other meal data) and the bolus calculator outputs a recommended size for the insulin bolus dosage. Although bolus calculators remove some of the mental calculations made by the user in determining an appropriate insulin bolus dosage, bolus calculators still burden the user with the mental task of evaluating the constituents of their meal, may require the use of a secondary device, and often require manual entry of data.

Although conventional dosing systems may remove some of the mental burdens for the PWD in determining an appropriate recommendation related to insulin dosing, dosing systems still burden the PWD with one or more of the mental tasks of manually evaluating therapy data, manually determining a dosing recommendation, manually determining injection sites, and manual entry of data.

### DISCLOSURE

The various embodiments described below provide benefits and/or solve one or more of the foregoing or other problems in the art with systems and methods for utilizing medicine delivery devices. Embodiments include a method of providing feedback to a user of a medicine delivery device. The method includes receiving information from one or more detectors of a pen cap of the medicine delivery device, analyzing the received information to identify at least one incorrect or correct usage of the medicine deliver device, and responsive to identifying the one or more of incorrect usages or correct usages, providing feedback via one or more feedback components of the pen cap of the medicine delivery device.

Some embodiments include a medicine delivery device. The medicine delivery device may include an injection pen and a pen cap. The pen cap may include one or more detectors, a vibration motor, and a controller. The controller may include at least one processor and at least one non-transitory computer-readable storage medium storing instructions thereon that, when executed by the at least one processor, cause the controller to: receive information from the one or more detectors of the pen cap regarding physical interactions between the pen cap and the injection pen, analyze the received information to identify at least one of an incorrect physical interaction or a correct physical interaction between the pen cap and the injection pen, and responsive to identifying the at least one of an incorrect physical interaction or a correct physical interaction between the pen cap and the injection pen, provide feedback via the vibration motor of the medicine delivery device.

One or more embodiments include a non-transitory computer-readable medium storing instructions thereon that, when executed by at least one processor, cause the at least one processor to perform steps comprising receiving information regarding physical interactions between a pen cap and an injection pen of a medicine delivery device, analyzing the received information to identify at least one incorrect physical interaction between the pen cap and the injection pen, and responsive to identifying the at least one incorrect physical interaction, providing haptic feedback via the pen cap.

### BRIEF DESCRIPTION OF THE DRAWINGS

Various embodiments will be described and explained with additional specificity and detail through the use of the accompanying drawings in which:

FIG. 1 shows a perspective view of a medicine delivery device in accordance with one or more embodiments; FIG. 2 shows a schematic representation of the medicine delivery device of FIG. 1 in accordance with one or more embodiments; FIG. 3 shows a flow chart of a method of providing feedback to a user regarding usage of a medicine delivery device according to one or more embodiments; and

FIG. 4 illustrates a block diagram of an exemplary computing device in accordance with one or more embodiments.

### MODE(S) FOR CARRYING OUT THE INVENTION

The illustrations presented herein are not actual views of any particular medicine delivery device, or any component thereof, but are merely idealized representations, which are employed to describe the present invention.

As used herein, the singular forms following “a,” “an,” and “the” are intended to include the plural forms as well, unless the context clearly indicates otherwise.

As used herein, the term “may” with respect to a material, structure, feature, function, or method act indicates that such is contemplated for use in implementation of an embodiment of the disclosure, and such term is used in preference to the more restrictive term “is” so as to avoid any implication that other compatible materials, structures, features, functions, and methods usable in combination therewith should or must be excluded.

As used herein, any relational term, such as “first,” “second,” etc., is used for clarity and convenience in understanding the disclosure and accompanying drawings, and does not connote or depend on any specific preference or order, except where the context clearly indicates otherwise.

As used herein, the term “substantially” in reference to a given parameter, property, act, or condition means and includes to a degree that one skilled in the art would understand that the given parameter, property, or condition is met with a small degree of variance, such as within acceptable manufacturing tolerances. By way of example, depending on the particular parameter, property, or condition that is substantially met, the parameter, property, or condition may be at least 90.0% met, at least 95.0% met, at least 99.0% met, or even at least 99.9% met.

As used herein, the term “about” used in reference to a given parameter is inclusive of the stated value and has the meaning dictated by the context (e.g., it includes the degree of error associated with measure of the given parameter, as well as variations resulting from manufacturing tolerances, etc.).

Embodiments of the present disclosure include a medicine delivery device having one or more detectors (e.g., sensors) for detecting interactions between a pen cap of the medicine delivery device and an injection pen (e.g., insulin pen) of the medicine delivery device and/or detecting interactions between the medicine delivery device and a user (e.g., dosing actions). Furthermore, the medicine delivery device includes one or more feedback components (e.g., haptic, visual, and/or audio feedback components) for providing feedback to the user regarding the interactions between the pen cap and the injection pen and/or interactions between the medicine delivery device and the user. For example, embodiments of the present disclosure include providing feedback via the one or more feedback components responsive to: the injection pen being inserted into the pen cap after administering medication, a new injection pen (e.g., insulin pen) being inserted into the pen cap, a correct injection pen being inserted into the pen cap, a mis-insertion of the injection pen into the pen cap, the injection pen being inserted into the pen cap with a needle and/or a sheath, and/or the injection pen being removed from the pen cap. Furthermore, the feedback may indicate via, for example, duration, amplitude, frequency, and/or cadence of the feedback, correct and/or incorrect interactions. For instance, the feedback may be specific to correct and/or incorrect interactions.

Accordingly, the medicine delivery devices described herein may provide advantages over conventional delivery devices. For instance, the medicine delivery devices of the present disclosure may improve a user experience in utilizing a medicine delivery device by providing confirmation of (e.g., feedback for) correct actions (e.g., correct usage) and notice (e.g., creating awareness) of incorrect actions (e.g., incorrect usage) when utilizing the medicine delivery device. As a non-limiting example, the medicine delivery devices may provide feedback through haptic feedback (e.g., vibration feedback), and the haptic feedback may create a more consistent user experience when inserting the injection pen into and removing the injection pen from a pen cap in comparison to conventional delivery devices. Moreover, a consistent user experience may reduce learning times and may reduce user confusion in comparison to conventional delivery devices.

For example, users of conventional delivery devices may experience various challenges in operating the delivery devices. One challenge a user may experience when using conventional delivery devices is “diabetes fatigue syndrome” (DFS). As used herein, DFS may be defined as a “multifactorial syndrome of fatigue or easy fatigability that occurs in persons with diabetes,” which may be caused by a variety of factors. Accordingly, decreasing learning obstacles and reducing confusion in using a delivery device may lessen a cognitive burden on the user and may improve the user experience. Another challenge a user may experience is neuropathy. A PWD often has nerve damage; accordingly, providing feedback via haptic feedback may be beneficial to a user whose fine motor skills and dexterity are limited. Likewise, haptic feedback may be beneficial to a user having hearing and/or vision impairments. In view of the foregoing, the medicine delivery device of the present disclosure may reduce a cognitive burden on the user by providing confirmation of correct actions and notice (e.g., creating awareness) of incorrect actions.

Embodiments of the disclosure include providing haptic feedback including vibrations of specified duration, amplitude, frequency, and/or cadence. Furthermore, parameters (e.g., haptic parameters) of the feedback may depend and/or be customized based on a medication therapy regime and/or a medication present (e.g., type of insulin) within the injection pen (e.g., present within a cartridge within the injection pen). The parameters of the feedback may be automatically and/or manually adjusted to address environmental and/or other challenges native to a user receiving the medication therapy regime (e.g., utilizing the medicine delivery device). For example, the parameters may depend and/or be customized based on a user profile of the user. For instance, depending on the user's fine motor skills, parameters such as length and amplitude of the haptic feedback may be adjusted to accommodate a user preference and/or capacities.

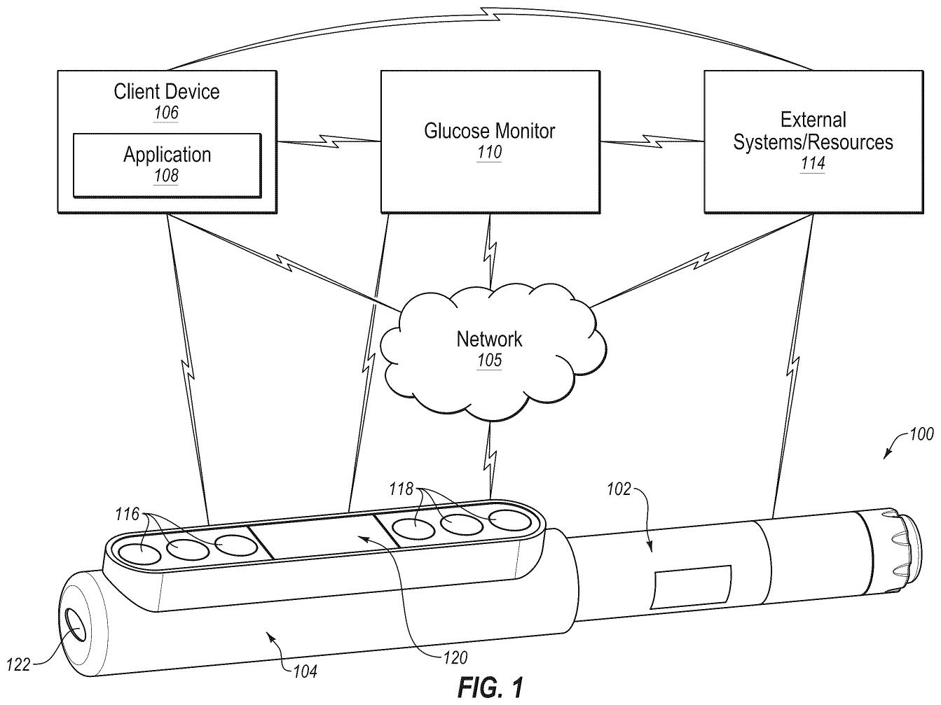

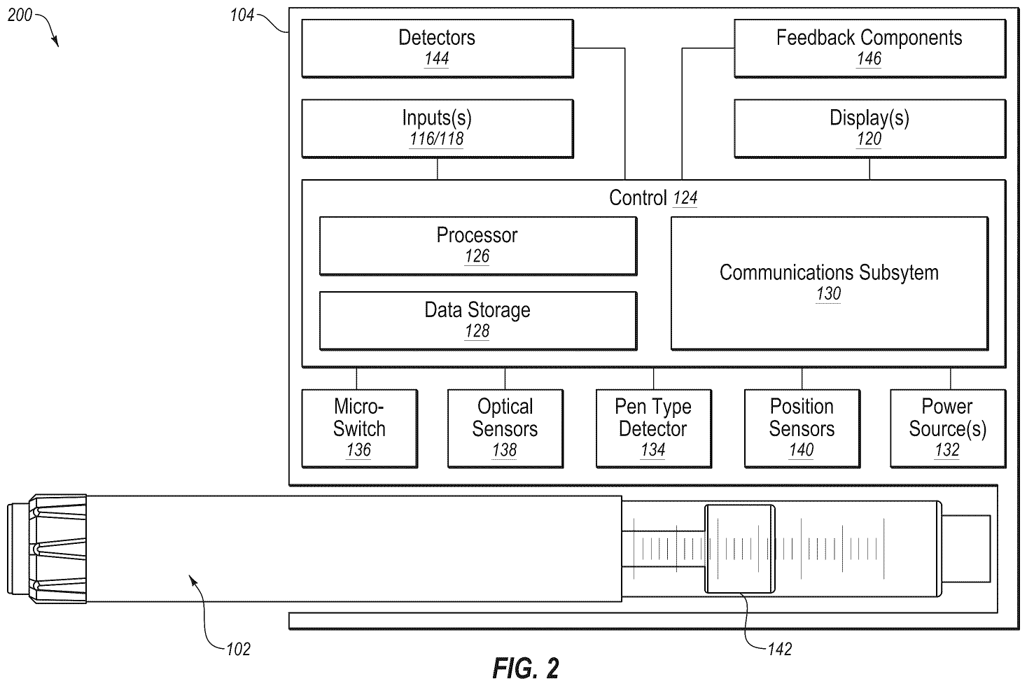

FIG. 1 is a perspective view of a medicine delivery device 100 according to one or more embodiments of the disclosure. As depicted in FIG. 1, in some embodiments, the medicine delivery device 100 may include an injection pen 102 (e.g., an insulin pen) and a pen cap 104. In some embodiments, the medicine delivery device 100 may include one or more of a quick acting insulin (QAI) pen or a long acting insulin (LAI) pen. As is discussed in greater detail below, the pen cap 104 may be in wireless communication with one or more of a client device 106, an application 108 of the client device 106, a glucose monitor 110, and/or the external systems/resources 114 via one or more networks 105.

In some embodiments, the application 108 (e.g., a tool application) of the client device 106 may include a medication therapy management system enabling users to manage their medication therapy and at least partially control and/or configure the medicine delivery device 100 (e.g., adjust settings of the medicine delivery device 100). As a non-limiting example, the application 108 may be directed to assisting a user in managing insulin therapy of the user. In some instances, the application 108 may be a web application for managing insulin therapy of the user. In some embodiments, the application 108 may be local to the client device 106. In other embodiments, the application 108 may be stored and/or at least partially operated via a cloud computing service. In additional embodiments, the application 108 may be stored and/or at least partially operated on the medicine delivery device 100. In some embodiments, the client device 106 may execute one or more applications (e.g., application 108) for performing the functions of the various embodiments and processes described herein.

In one or more embodiments, the application 108 may be a native application installed on the client device 106. For example, the application 108 may be a mobile application that installs and runs on a mobile device, such as a smart phone or a tablet. The application 108 may be specific to an operating system of the client device 106. Further, in some embodiments, the application 108 may be a client application that is associated with the medication therapy management system and/or at least a portion of the medicine delivery device 100 (e.g., the pen cap 104 of the medicine delivery device 100) and configured to enable interaction directly with the medication therapy management system through the application 108.

The client device 106, the glucose monitor 110, the one or more external systems/resources 114, and the medicine delivery device 100 may communicate via the one or more networks 105. In one or more embodiments, the one or more networks 105 may include a combination of cellular or mobile telecommunications networks, a public switched telephone network (PSTN), and/or the Internet or World Wide Web that facilitate the transmission of data between the client device 106 (e.g., the injection site determination system 106), the glucose monitor 110, the one or more external systems/resources 114, and the medicine delivery device 100. The network 105, however, may include various other types of networks that use various communication technologies and protocols, such as a wireless local network (WLAN), a wide area network (WAN), a metropolitan area network (MAN), other telecommunication networks, or a combination of two or more of the foregoing networks. In additional embodiments, the client device 106, the glucose monitor 110, the one or more external systems/resources 114, and the medicine delivery device 100 may communicate via Bluetooth and Near-field communication in addition to or instead of the one or more networks 105.

Although FIG. 1 illustrates a particular arrangement of the client device 106, the glucose monitor 110, the one or more external systems/resources 114, the one or more networks 105, and the medicine delivery device 100, various additional arrangements are possible. For example, the medicine delivery device 100, the glucose monitor 110, and/or the one or more external systems/resources 114 may directly communicate with the client device 106 bypassing the network 105.

A user may interface with the client device 106, for example, to utilize the medication therapy management system in order to input user preferences, adjust profiles, view notifications, interact with a provider, adjust settings of the medicine delivery device 100, etc.

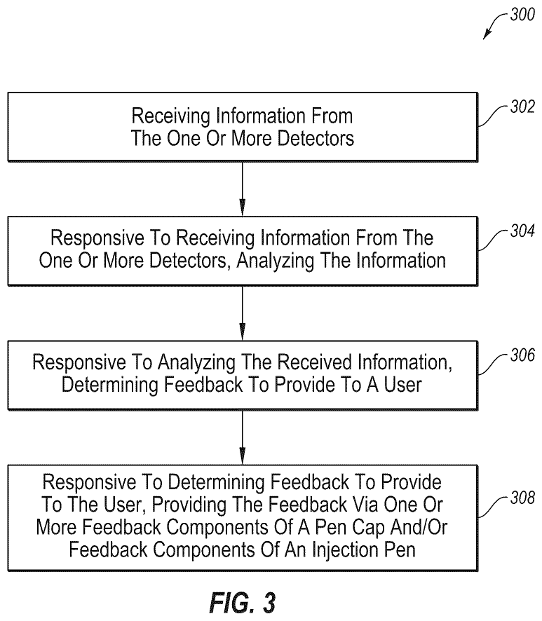

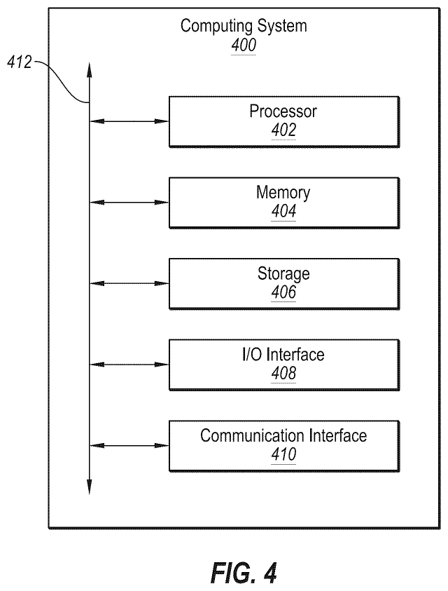

The client device 106 may be any one or more of various types of computing devices. For example, the client device 106 may include a mobile device such as a mobile telephone, a smartphone, a PDA, a tablet, or a laptop, or a non-mobile device such as a desktop or another type of computing device. Additional details with respect to the client device 106 are discussed below with respect to FIG. 4.

The external systems/resources 114 may include additional systems that interface with the client device 106, the application 108, the medication therapy management system, and/or the medicine delivery device. The external systems/resources 114, in some embodiments, may include additional medical devices. The medical devices may include additional insulin delivery systems, including without limitation, insulin delivery devices (e.g., infusion pumps, injection pens, and inhalers), glucose sensors (e.g., CGMs and blood glucose meters), therapy managers (e.g., controllers for controlling open and closed-loop delivery of insulin or aspects of delivering insulin and recommendation systems for providing therapy recommendations to users and/or health providers), and combinations thereof. In some embodiments, the external systems/resources 114 may include subject matter expert input data, clinical literature, conventional medication regimes, etc. The external systems/resources 114, in various embodiments, may include a therapy management system(s). Therapy management systems may include a diabetes management system for monitoring blood glucose data and therapy data and managing therapy settings. In further embodiments, the external systems/resources 114 may include health care provider devices. In additional embodiments, the external systems/resources 114 may include a cloud computing platform and/or one or more servers. Furthermore, medication therapy management system may be at least partially operated on the external systems/resources 114.

In some embodiments, the glucose monitor 110 may include any known glucose monitor. For example, the glucose monitor may include one or more of a continuous glucose monitor (CGM), a flash glucose monitor, a blood glucose meter (BGM), or any other suitable sensor. In the case of CGMs and flash glucose monitors, the CGMs and flash glucose monitor may provide glucose data based on interstitial fluid glucose levels of a user, which may be correlated to blood glucose levels. A BGM may be configured to provide blood glucose data, typically based on a blood sample. Accordingly, the term “blood glucose” is not limited to using just blood glucose data, values, levels, etc., but it also includes interstitial fluid glucose levels, as well as any intermediate measurement values.

FIG. 2 is a schematic representation of the medicine delivery device 100 of FIG. 1 including the pen cap 104 and the injection pen 102. Referring to FIGS. 1 and 2 together, the pen cap 104 may include a display screen 120, one or more inputs 116 (e.g., dials, buttons, and/or touch screen regions) for a user to set a dosage to be delivered, one or more inputs 118 for inputting meals information, inputting insulin dose information, responding to recommendations, etc., one or more indicator lights 122, which may light up to indicate that it is transferring data, light up to indicate that the user's attention is needed, and/or light up to indicate whether a dose capture functionality is or is not working. Additionally, the pen cap 104 may utilize the display screen 120 for displaying one or more of an estimated glucose value (EVG), units for the EVG, a trend indicator for the EVG, a recommended dosage, an identification of the type of insulin, a recommended site injection, a time and amount of a previous dosage, and/or an insulin on board value to remind a user about their most recent dosage. In some embodiments, the display screen 120 of the pen cap 104 may include a touch screen, which may include the one or more inputs 116 and/or the one or more inputs 118. Additionally, the pen cap 104 may itself include dose-capture technology.

Referring still to FIGS. 1 and 2 together, the pen cap 104 may include a controller 124 including a processor 126, data storage 128 (or memory), and a communications subsystem 130. The communications subsystem 130 may enable wireless communication between the pen cap 104 and the client device 106 and/or a glucose monitor 110. In some instances, the communications subsystem 130 may include a near field communications (NFC) chip. In some instances, the communications subsystem 130 may include a Bluetooth Low Energy (BLE) chip. In some instances, the communications subsystem 130 may include an optical communication device, an infrared communication device, a wireless communication device (such as an antenna), and/or chipset (such as a Bluetooth device (e.g., Bluetooth Low Energy, Classic Bluetooth, etc.), a Near-field communication (NFC) device, an 802.6 device (e.g., Metropolitan Area Network (MAN), a Zigbee device, etc.), a WiFi device, a WiMax device, cellular communication facilities, etc.), and/or the like. In these and other cases, the communications subsystem 130 may exchange data with a network and/or any other device or system described in the present disclosure.

Additionally, in some embodiments, the pen cap 104 may include a power source 132, which may include a rechargeable or non-rechargeable battery. Furthermore, the pen cap 104 may include a pen type detector 134, a micro switch 136, optical sensor(s) 138, and position sensor(s) 140. In one or more embodiments, the controller 124 may determine a pen type from data from the pen type detector 134 The controller 124 may also determine a position of a plunger 142 within the injection pen 102 using one or more of the micro switch 136, the optical sensor(s) 138, and position sensor(s) 140. Based on the determined positions of the plunger 142, dosing events and/or amounts of insulin delivered may be determined by the controller 124.

Furthermore, the pen cap 104 may include one or more sensors/detectors 144 (referred to hereinafter as “one or more detectors 144”) operably coupled to the controller 124 and to determine and identify user interactions with the pen cap 104, a status of the pen cap 104, interactions between the pen cap 104 and the injection pen 102, and/or orientations of the pen cap 104 relative to the injection pen 102 and vis-a-versa. For example, the one or more detectors 144 may include one or more of an injection pen entry detector, an injection pen insertion level detector, an injection pen pull force detector, an accelerometer, a temperature sensor, or a clasping force detector.

The injection pen entry detector may be disposed at an opening of the pen cap 104 configured to receive the injection pen 102. In some embodiments, the injection pen entry detector may include one or more of a proximity sensor, an infrared sensor, or an image sensor. In one or more embodiments, the injection pen entry detector may include a no-contact sensor. The injection pen entry detector may be utilized to detect entry and/or removal of an injection pen 102 and to provide information (e.g., data) to the controller 124 regarding whether an injection pen 102 is being inserted into and/or removed from the pen cap 104. In some embodiments, the proximity sensor may include one or more optical proximity sensors, sound proximity sensors, magnetic proximity sensors, and/or capacitive proximity sensors. Furthermore, the infrared sensors may include one or more of a transmissive or reflective type infrared sensor. The image sensors may include one or more of a charged coupled device sensor or a CMOS image sensor.

The injection pen insertion level detector may be disposed proximate an end (e.g., a bottom) of the pen cap 104 opposite the opening of the pen cap 104. In some embodiments, the injection pen insertion level detector may include one or more of a proximity sensor or a force sensor. The injection pen insertion level detector may be utilized to detect when an injection pen 102 reaches the end of the pen cap 104 opposite the opening of the pen cap 104 and to provide information (e.g., data) to the controller 124 regarding when an injection pen 102 has be inserted into the pen cap 104 to an extent where a portion of the injection pen 102 has reached the end of the pen cap 104 opposite the opening of the pen cap 104 (e.g., fully inserted into the pen cap 104). The proximity sensors may include any of the proximity sensors described above. The force sensor may include one or more of a load cell, a strain gauge, a pressure sensor, or any other known force sensor.

The injection pen pull force detector may be disposed within one or more portions (e.g., a clasp, a radial bump, frictional clasp, etc.) of the pen cap 104 configured to grasp (e.g., clasp, engage, etc.) an injection pen 102 via mechanical force when the injection pen 102 is disposed within the pen cap 104. In one or more embodiments, the injection pen pull force detector may include a force sensor. The injection pen pull force detector may be utilized to determine an amount of force the injection pen 102 is experiencing at a given moment and to provide information (e.g., data) to the controller 124 regarding forces being experienced by the injection pen 102. In some embodiments, the injection pen pull force detector may be configured to determine an amount of force the injection pen 102 is experiencing in a direction parallel to a longitudinal axis of the pen cap 104. The force sensor may include any of the force sensors described above. In some embodiments, data from the injection pen pull force detector may be utilized as a safety mechanism to enable a user to remove the injection pen 102 from the pen cap 104 with a clasping mechanism being in an engaged state (i.e., failure of operation of the clasping mechanism).

The clasping force detector may be disposed within one or more portions of the pen cap 104 configured to grasp (e.g., a clasp, engage, etc.) an injection pen 102 via mechanical force when the injection pen 102 is disposed within the pen cap 104. In one more embodiments, the clasping force detector may include a force sensor. The clasping force detector may be utilized to determine an amount of force that the pen cap 104 is applying to the injection pen 102 at a given moment and to provide information (e.g., data) to the controller 124 regarding forces being applied to the injection pen 102. In some embodiments, the clasping force detector may be configured to determine an amount and a direction of forces the pen cap 104 (e.g., a clasping mechanism of the pen cap 104) is applying to the injection pen 102. The force sensor may include any of the force sensors described above.

The accelerometer may be disposed within any portion of the pen cap 104. The accelerometer may be utilized to identify movement and/or orientation of the pen cap 104 and to provide information (e.g., data) to the controller regarding movement and/or orientation of the pen cap 104. The information received from accelerometer may be utilized in operation of waking features, sleep modes, and power consumption of the pen cap 104.

The temperature sensor may be disposed within any portion of the pen cap 104. The temperature sensor may be utilized to monitor the temperature of the pen cap 104, the injection pen 102, or both and to provide information (e.g., data) to the controller regarding the temperature sensor of the pen cap 104. The information received from temperature sensor may be utilized in determining medication viability, optimizing battery charging, and determining usage patterns.

Referring still to FIGS. 1 and 2, the pen cap 104 may further include one or more feedback components 146 for providing feedback to a user. The one or more feedback components 146 may be disposed within or on any portion of the pen cap 104 and may be operably coupled to the controller 124. The one or more feedback components 146 may include one or more of a vibration motor (e.g., solenoid), an audio transducer, or a display (e.g., display screen 120). As is described in greater detail below, the feedback components 146 may be utilized to provide feedback to a user to indicate correct user interactions with the pen cap 104, proper statuses of the pen cap 104, correct interactions between the pen cap 104 and the injection pen 102 (e.g., a correct insertion of the injection pen 102 into the pen cap 104), and/or correct orientations of the pen cap 104 relative to the injection pen 102 and vis-a-versa and/or to indicate incorrect user interactions with the pen cap 104, improper statuses of the pen cap 104, incorrect interactions between the pen cap 104 and the injection pen 102 (e.g., an incorrect insertion of the injection pen 102 into the pen cap 104), and/or incorrect orientations of the pen cap 104 relative to the injection pen 102. Additionally, the feedback components 146 may be utilized to provide feedback to a user regarding scans of a continuous glucose monitor or blood glucose meter, battery levels, temperatures, viability of medicine within the injection pen 102, an incorrect injection pen insertion, and/or insertion of a foreign object.

The vibration motor may include any conventional vibration motor (e.g., haptic solenoid) and may be configured to provide haptic feedback to a user. In some embodiments, the vibration motor may include a designated motor. In additional embodiments, the vibration motor may be a part of another element of the pen cap 104, such as an actuation motor for another operation of the pen cap 104.

The audio transducer may include one or more of audio speakers or piezoelectric transducers.

The display may include any conventional display for providing (e.g., displaying) information. For example, the display may include one or more of electronic paper (e.g., electronic ink, e-ink, or electrophoretic display), a liquid crystal display (LCD) screen, a light-emitting diode (LED) screen, or any other conventional display.

FIG. 3 shows a flow chart of a method 300 of providing feedback to a user regarding usage of a medicine delivery device. Referring to FIGS. 1-3 together, the method 300 may include receiving information (e.g., data) from the one or more sensors/detectors 144, as shown in act 302 of FIG. 3. For example, the controller 124 may receive information from one or more of an injection pen entry detector, an injection pen insertion level detector, an injection pen pull force detector, an accelerometer, a temperature sensor, or a clasping force detector, such as any of the detectors 144 described above. In some embodiments, the controller 124 may receive the information via one or more of wireless or wired connections. In one or more embodiments, the controller 124 may provide at least a portion of the received information to the client device 106.

In some embodiments, the controller 124 may receive the information responsive to an event. For example, the controller 124 may receive the information responsive to a user interaction with one or more of the injection pen 102 or the pen cap 104 (e.g., one or more of the detectors 144 detecting a user interaction). The user interaction may include one or more of a user inserting the injection pen 102 into the pen cap 104 (e.g., a capping event), a user removing the injection pen 102 from the pen cap 104 (e.g., an uncapping event), or a dosing action or event. The dosing actions and dosing events may include any of the dosing actions and dosing events described in U.S. application Ser. No. 16/442,281, to Bowland et. al., filed Jun. 14, 2019, and/or U.S. Pat. No. 11,154,660 B2, to Sjolund et. al., filed Dec. 12, 2018, the disclosures of which are incorporated in their entireties by reference herein. In some embodiments, the controller 124 may receive the information responsive to a time elapsing since a user interaction (e.g., event). In one or more embodiments, the controller 124 may receive the information responsive to a measured temperature. In some embodiments, the controller 124 may continuously receive the information.

In one or more embodiments, the information received from the detectors 144 may include one or more of an indication of entry and/or removal of an injection pen 102 from the pen cap 104, an indication of when an injection pen 102 reaches the end of the pen cap 104 opposite the opening of the pen cap 104, an indication of an amount of force the injection pen 102 is experiencing at a given moment, and indication of an amount of force that the pen cap 104 is applying to the injection pen 102 at a given moment, an indication of movement and/or an orientation of the pen cap 104, and/or an indication of a temperature of the pen cap 104.

Responsive to receiving information (e.g., data) from the one or more sensors/detectors 144, the method 300 may include analyzing the information, as shown in act 304 of FIG. 3. For example, the controller 124 may analyze the received information. In additional embodiments, the controller 124 may provide the received information to a server (e.g., external systems/resources 114) and/or the client device 106 (e.g., mobile phone), and the server and/or the client device 106 may analyze the received information. In some embodiments, analyzing the received information may include identifying correct usages and/or incorrect usages of the injection pen 102 and/or the pen cap 104. As used herein, the term “usage” may refer to capping the injection pen 102 with the pen cap 104, uncapping the pen cap 104 from the injection pen 102, administering medication via the injection pen 102 and pen cap 104, or any other usages of a pen cap or an injection pen typical when performing medicine (e.g., insulin) therapy. Additionally, “usage” may refer to insertion of the injection pen 102 into the pen cap 104 subsequent to administering medication, insertion of a new injection pen into the pen cap 104, and insertion of the injection pen 102 with or without a needle and/or a sheath into the pen cap 104. As noted above, identifying usages of the injection pen 102 and/or the pen cap 104 may include identifying correct usages and/or incorrect usages of the injection pen 102 and/or the pen cap 104.

As a non-limiting example, the controller 124 may determine whether the injection pen 102 has been correctly or incorrectly inserted into the pen cap 104. In some embodiments, the controller 124 may utilize information from one or more of the injection pen entry detector, the injection pen insertion level detector, and the clasping force detector to determine whether the injection pen 102 has been correctly or incorrectly inserted into the pen cap 104. For example, the controller 124 may determine, via the injection pen entry detector and/or the injection pen insertion level detector, whether the injection pen 102 has been sufficiently inserted into the pen cap 104 (e.g., whether a longitudinal end of the injection pen 102 is within a predetermined distance of the end of the pen cap 104). Additionally, the controller 124 may determine, via the clasping force detector, whether a force being applied to the injection pen 102 is within a predetermined range (e.g., appropriate range).

Responsive to determining that the injection pen 102 has been sufficiently inserted into the pen cap 104 and that a force being applied to the injection pen 102 is within a predetermined appropriate range, the controller 124 may determine that the injection pen 102 has been properly inserted into the pen cap 104 and constitutes a correct usage (e.g., interaction). Responsive to determining that the injection pen 102 has been insufficiently inserted into the pen cap 104 or that a force being applied to the injection pen 102 is outside of a predetermined appropriate range, the controller 124 may determine that the injection pen 102 has been improperly inserted into the pen cap 104 and constitutes an incorrect usage (e.g., interaction).

As another non-limiting example, the controller 124 may determine whether capping or uncapping events of the pen cap 104 appropriately align with a scheduled medication dosing action and/or a detected medication dosing action. For instance, the controller 124 may determine whether or not the pen cap 104 has been uncapped from the injection pen 102 prior to or at a scheduled medication dosing action. Additionally, the controller 124 may determine whether or not the pen cap 104 has been capped onto the injection pen subsequent to a scheduled and/or detected medication dosing action. In some embodiments, the controller 124 may determine how long the pen cap 104 was uncapped from the injection pen 102 and whether the amount of time the pen cap 104 was uncapped from the injection pen 102 was a sufficient time to perform a medication dosing action.

Responsive to determining that capping and/or uncapping events of the pen cap 104 appropriately align with a scheduled and/or a detected medication dosing action, the controller 124 may determine or infer that a medication dosing action appropriately occurred and constitutes a correct usage (e.g., interaction). Responsive to determining that capping and/or uncapping events of the pen cap 104 do not properly align with a scheduled and/or a detected medication dosing action, the controller 124 may determine or infer that a medication dosing action did not occur and constitutes an incorrect usage (e.g., interaction).

As noted above, the controller 124 may also determine, via one or more of the detectors 144, (1) when a new injection pen 102 is inserted into the pen cap 104, (2) when a correct or incorrect injection pen 102 is inserted into the pen cap 104, (3) when the injection pen 102 is inserted into the pen cap 104 with or without a needle and/or sheath, (4) orientations of the injection pen 102 and/or the pen cap 104, and (5) temperatures experienced by the injection pen 102 and/or the pen cap 104. Furthermore, for each of the foregoing examples, the controller 124 may identify the usages (e.g., interactions) relative to detected or scheduled dosing actions. Moreover, for each of the foregoing examples, the controller 124 may determine whether the usage (e.g., interaction) was correct or incorrect.

Responsive to analyzing the received information, the method 300 may include determining feedback to provide to a user, as shown in act 306 of FIG. 3. For example, the controller 124 may utilize the analysis performed above in regard to act 304 of FIG. 3 to determine feedback to provide to the user. For instance, as is discussed in further detail below, the controller 124 may determine feedback to provide to the user based at least partially on whether the analysis indicated correct and/or incorrect usages of the injection pen 102 and/or the pen cap 104.

In some embodiments, determining feedback to provide to the user may include determining whether feedback to the user is warranted (e.g., necessary or appropriate) given the analysis of the received information. For example, if incorrect usages are identified via the analysis, the controller 124 may determine that feedback is appropriate to bring awareness to the incorrect usages, indicate corrective action is needed, and/or as an alarm. As another non-limiting example, if correct usages are identified via the analysis, the controller 124 may determine that feedback is appropriate to indicate correct usage to a user (e.g., that the user performed an action (e.g., capping and/or dosing action) correctly) and/or as positive reinforcement of an action. In some embodiments, if correct routine usages are identified via the analysis, the controller 124 may determine that feedback is not warranted or necessary. In some embodiments, determining whether feedback to the user is warranted may be based at least partially on a user profile and/or user preferences (e.g., previously made selections).

In some embodiments, only certain usages (e.g., actions) are categorized as warranting feedback. For example, in one or more embodiments, usages determined and/or categorized (e.g., categorized by a provider) as critical or necessary to an insulin therapy regime of the user may be categorized as warranting feedback. For instance, dosing actions, capping actions, and/or uncapping actions may be categorized as warranting feedback.

In one or more embodiments, determining feedback to provide to the user may include determining a type of feedback to provide to the user. For example, the controller 124 may determine to provide types of feedback such as haptic, audible, and/or visual feedback. Moreover, responsive to determining a type of feedback to provide to the user, the controller 124 may determine a form of the feedback. For example, in regard to haptic feedback, the controller 124 may determine one or more of an intensity, a duration, an amplitude, a frequency, and/or cadence of haptic feedback. In regard to audible feedback, the controller 124 may determine one or more of a volume, a sound, a duration, an amplitude, a frequency, and/or a cadence of audible feedback. In regard to visual feedback, the controller 124 may determine one or more of a message, an icon, a shape, and/or an appearance of the visual feedback.

In some embodiments, the types and forms of the feedback may be preselected and customizable via user settings, a user profile, and/or provider settings, and the types and forms of the feedback may be customized within a medication therapy regime (e.g., insulin therapy regime) and to accommodate a user preference and/or capacities.

Responsive to determining feedback to provide to the user, the method 300 may include providing the feedback via the one or more feedback components 146 of the pen cap 104 and/or feedback components of the injection pen 102, as shown in act 308 of FIG. 3. For example, the controller 124 may cause the one or more feedback components 146 of the pen cap 104 to output the feedback determined in act 306 of FIG. 3. As a non-limiting example, the controller 124 may cause one or more of a vibration motor, an audio transducer, or a display (e.g., display screen 120) of the pen cap 104 and/or feedback components of the injection pen 102 to output the feedback.

As a non-limiting example, in embodiments where the one or more feedback components 146 of the pen cap 104 include a vibration motor, the controller 124 may cause the vibration motor to vibrate according to the determined form of the feedback. In embodiments where the one or more feedback components 146 of the pen cap 104 an audio transducer, the controller 124 may cause the audio transducer to output sound according to the determined form of the feedback. In embodiments where the one or more feedback components 146 of the pen cap 104 a display (e.g., display screen 120), the controller 124 may cause the display to display one or more messages, icons, lights, etc., according to the determined form (e.g., visual appearance) of the feedback. Additionally, the controller 124 may cause the one or more feedback components 146 of the pen cap 104 to provide feedback via any combination of the manners described above. In some embodiments, the controller 124 may further cause feedback components of the injection pen 102 to provide feedback via any combination of the manners described above. For instance, the controller 124 may cause the feedback components 146 of the pen cap 104 to output two or more types of feedback (e.g., both haptic and audible feedback, both haptic and visual feedback, both audible and visual feedback).

In some embodiments, the medicine delivery device 100 (e.g., the pen cap 104) may transfer data to the client device 106 (e.g., a mobile device) with instructions to provide the determined feedback or one or more portions of the determined feedback. In one or more embodiments, the medicine delivery device 100 (e.g., the pen cap 104) may transfer data to the client device 106 with instructions to provide the determined feedback in addition to feedback output by the medicine delivery device 100 or instead of the feedback output by the medicine delivery device 100.

Referring to FIGS. 1-3 together, the medicine delivery device 100, one or more detectors 144, and one or more feedback components 146 (referred to hereinafter collectively as “the medicine delivery device 100”) described herein may provide advantages over conventional delivery devices. For instance, the medicine delivery devices 100 of the present disclosure may improve a user experience in utilizing a medicine delivery device 100 by providing confirmation of (e.g., feedback for) correct actions (e.g., correct usage) and notice (e.g., creating awareness) of incorrect actions (e.g., incorrect usage) when utilizing the medicine delivery device 100. As discussed above, the medicine delivery device 100 may provide feedback through haptic feedback (e.g., vibration feedback), and the haptic feedback may create a more consistent user experience when inserting the injection pen 102 into and removing the injection pen 102 from a pen cap 104 in comparison to conventional delivery devices. Moreover, a consistent user experience may reduce learning times and may reduce user confusion in comparison to conventional delivery devices.

For example, users of conventional delivery devices may experience various challenges in operating the delivery devices. One challenge a user may experience when using conventional delivery devices is “diabetes fatigue syndrome” (DFS). As used herein, DFS may be defined as a “multifactorial syndrome of fatigue or easy fatigability that occurs in persons with diabetes” which may be caused by a variety of factors. Accordingly, decreasing learning obstacles and reducing confusion in using a delivery device may lessen a cognitive burden on the user and may improve the user experience. Another challenge a user may experience is neuropathy. A PWD often has nerve damage; accordingly, providing feedback via haptic feedback may be beneficial to a user whose fine motor skills and dexterity are limited. Likewise, haptic feedback may be beneficial to a user having hearing and/or vision impairments. In view of the foregoing, the medicine delivery device 100 of the present disclosure may reduce a cognitive burden on the user by providing confirmation of correct actions and notice (e.g., creating awareness) of incorrect actions.

Referring still to FIGS. 1-3 together, any of the above-described feedback may occur responsive to different interactions between the injection pen 102 and the pen cap 104. For example, any of the above-described feedback may occur responsive to the injection pen 102 being inserted into the pen cap 104 after administering insulin, when a new injection pen 102 (e.g., insulin pen) is inserted into the pen cap 104, when correct injection pen 102 is inserted into the pen cap 104, when there is a mis-insertion of the injection pen 102 into the pen cap 104, when the injection pen 102 is inserted into the pen cap 104 with a needle and/or a sheath, and/or when the injection pen 102 is removed from the pen cap 104.

The feedback described herein may include a vibration of specified duration, amplitude, frequency, and/or cadence. Furthermore, parameters (e.g., haptic parameters) of the feedback may depend and/or be customized based on a medication therapy regime and/or a medication present (e.g., type of insulin) within the injection pen 102 (e.g., present within a cartridge within the injection pen 102). The parameters of the feedback may be automatically and/or manually adjusted to address environmental and/or other challenges native to a user receiving the medication therapy regime (e.g., utilizing the medicine delivery device). For example, the parameters may depend and/or be customized based on a user profile of the user. For instance, depending on the user's fine motor skills, parameters such as length and amplitude of the haptic feedback may be adjusted to accommodate a user preference and/or capacities.

FIG. 4 is a block diagram of an exemplary computing device 400 that may be utilized as a client device (e.g., client device 106) or within and/or as a portion of the medicine delivery device 100 that may be configured to perform one or more of the processes described above. One will appreciate that one or more computing devices may implement the computing device 400. The computing device 400 may comprise a processor 402, a memory 404, a storage device 406, an I/O interface 408, and a communication interface 410, which may be communicatively coupled by way of a communication infrastructure 412. While an exemplary computing device is shown in FIG. 4, the components illustrated in FIG. 4 are not intended to be limiting. Additional or alternative components may be used in other embodiments. Furthermore, in certain embodiments, the computing device 400 may include fewer components than those shown in FIG. 4. Components of the computing device 400 shown in FIG. 4 will now be described in additional detail.

In one or more embodiments, the processor 402 includes hardware for executing instructions, such as those making up a computer program. As an example, and not by way of limitation, to execute instructions, the processor 402 may retrieve (or fetch) the instructions from an internal register, an internal cache, the memory 404, or the storage device 406 and decode and execute them. In one or more embodiments, the processor 402 may include one or more internal caches for data, instructions, or addresses. As an example, and not by way of limitation, the processor 402 may include one or more instruction caches, one or more data caches, and one or more translation lookaside buffers (TLBs). Instructions in the instruction caches may be copies of instructions in the memory 404 or the storage device 406.

The memory 404 may be used for storing data, metadata, and programs for execution by the processor(s). The memory 404 may include one or more of volatile and non-volatile memories, such as Random Access Memory (“RAM”), Read-Only Memory (“ROM”), a solid state disk (“SSD”), Flash memory, Phase Change Memory (“PCM”), or other types of data storage. The memory 404 may be internal or distributed memory.

The storage device 406 includes storage for storing data or instructions. As an example, and not by way of limitation, storage device 406 may comprise a non-transitory storage medium described above. The storage device 406 may include a hard disk drive (HDD), a floppy disk drive, flash memory, an optical disc, a magneto-optical disc, magnetic tape, or a Universal Serial Bus (USB) drive or a combination of two or more of these. The storage device 406 may include removable or non-removable (or fixed) media, where appropriate. The storage device 406 may be internal or external to the computing device 400. In one or more embodiments, the storage device 406 is non-volatile, solid-state memory. In other embodiments, the storage device 406 includes read-only memory (ROM). Where appropriate, this ROM may be mask programmed ROM, programmable ROM (PROM), erasable PROM (EPROM), electrically erasable PROM (EEPROM), electrically alterable ROM (EAROM), or flash memory or a combination of two or more of these.

The I/O interface 408 allows a user to provide input to, receive output from, and otherwise transfer data to and receive data from computing device 400. The I/O interface 408 may include a mouse, a keypad or a keyboard, a touch screen, a camera, an optical scanner, network interface, modem, other known I/O devices or a combination of such I/O interfaces. The I/O interface 408 may include one or more devices for presenting output to a user, including, but not limited to, a graphics engine, a display (e.g., a display screen), one or more output drivers (e.g., display drivers), one or more audio speakers, and one or more audio drivers. In certain embodiments, the I/O interface 408 is configured to provide graphical data to a display for presentation to a user. The graphical data may be representative of one or more graphical user interfaces and/or any other graphical content as may serve a particular implementation.

The communication interface 410 may include hardware, software, or both. In any event, the communication interface 410 may provide one or more interfaces for communication (such as, for example, packet-based communication) between the computing device 400 and one or more other computing devices or networks. As an example, and not by way of limitation, the communication interface 410 may include a network interface controller (NIC) or network adapter for communicating with an Ethernet or other wire-based network or a wireless NIC (WNIC) or wireless adapter for communicating with a wireless network, such as a WI-FI.

Additionally or alternatively, the communication interface 410 may facilitate communications with an ad hoc network, a personal area network (PAN), a local area network (LAN), a wide area network (WAN), a metropolitan area network (MAN), or one or more portions of the Internet or a combination of two or more of these. One or more portions of one or more of these networks may be wired or wireless. As an example, the communication interface 410 may facilitate communications with a wireless PAN (WPAN) (such as, for example, a BLUETOOTH®WPAN), a WI-FI network, a WI-MAX network, a cellular telephone network (such as, for example, a Global System for Mobile Communications (GSM) network), or other suitable wireless network or a combination thereof.

Additionally, the communication interface 410 may facilitate communications various communication protocols. Examples of communication protocols that may be used include, but are not limited to, data transmission media, communications devices, Transmission Control Protocol (“TCP”), Internet Protocol (“IP”), File Transfer Protocol (“FTP”), Telnet, Hypertext Transfer Protocol (“HTTP”), Hypertext Transfer Protocol Secure (“HTTPS”), Session Initiation Protocol (“SIP”), Simple Object Access Protocol (“SOAP”), Extensible Mark-up Language (“XML”) and variations thereof, Simple Mail Transfer Protocol (“SMTP”), Real-Time Transport Protocol (“RTP”), User Datagram Protocol (“UDP”), Global System for Mobile Communications (“GSM”) technologies, Code Division Multiple Access (“CDMA”) technologies, Time Division Multiple Access (“TDMA”) technologies, Short Message Service (“SMS”), Multimedia Message Service (“MMS”), radio frequency (“RF”) signaling technologies, Long Term Evolution (“LTE”) technologies, wireless communication technologies, in-band and out-of-band signaling technologies, and other suitable communications networks and technologies.

The communication infrastructure 412 may include hardware, software, or both that couples components of the computing device 400 to each other. As an example and not by way of limitation, the communication infrastructure 412 may include an Accelerated Graphics Port (AGP) or other graphics bus, an Enhanced Industry Standard Architecture (EISA) bus, a front-side bus (FSB), a HYPERTRANSPORT (HT) interconnect, an Industry Standard Architecture (ISA) bus, an INFINIBAND interconnect, a low-pin-count (LPC) bus, a memory bus, a Micro Channel Architecture (MCA) bus, a Peripheral Component Interconnect (PCI) bus, a PCI-Express (PCIe) bus, a serial advanced technology attachment (SATA) bus, a Video Electronics Standards Association local (VLB) bus, or another suitable bus or a combination thereof.

Embodiments of the present disclosure further include:

Embodiment 1. A method of providing feedback to a user of a medicine delivery device, comprising: receiving information from one or more detectors of a pen cap of the medicine delivery device; analyzing the received information to identify at least one of an incorrect usage or a correct usage of the medicine deliver device; and responsive to identifying the one or more of incorrect usages or correct usages, providing feedback via one or more feedback components of the pen cap of the medicine delivery device.

Embodiment 2. The method of embodiment 1, wherein identifying at least one of an incorrect usage or a correct usage comprises identifying physical interactions between the pen cap and an injection pen of the medicine delivery device.

Embodiment 3. The method of any one of embodiments 1 or 2, wherein identifying at least one of an incorrect usage or a correct usage comprises identifying a capping event between the pen cap of the medicine delivery device and an injection pen of the medicine delivery device.

Embodiment 4. The method of any one of embodiments 1-3 wherein identifying at least one of an incorrect usage or a correct usage comprises identifying an uncapping event between the pen cap of the medicine delivery device and an injection pen of the medicine delivery device.

Embodiment 5. The method of any one of embodiments 1-4, wherein identifying at least one of an incorrect usage or a correct usage comprises identifying insertion of a new injection pen into the pen cap of the medicine delivery device.

Embodiment 6. The method of any one of embodiments 1-5, wherein identifying at least one of an incorrect usage or a correct usage comprises identifying an incomplete insertion of an injection pen of the medicine delivery device into the pen cap of the medicine delivery device.

Embodiment 7. The method of any one of embodiments 1-6, wherein identifying at least one of an incorrect usage or a correct usage comprises identifying a complete insertion of an injection pen of the medicine delivery device into the pen cap of the medicine delivery device.

Embodiment 8. The method of any one of embodiments 1-7, wherein identifying at least one of an incorrect usage or a correct usage comprises identifying insertion of an injection pen of the medicine delivery device with at least one of a needle or a sheath into the pen cap of the medicine delivery device.

Embodiment 9. The method of any one of embodiments 1-8, wherein receiving information from one or more detectors of the pen cap of the medicine delivery device comprises receiving information from an injection pen entry detector.

Embodiment 10. The method of embodiment 9, wherein receiving information from the injection pen entry detector comprises receiving information regarding entry of an injection pen into the pen cap or removal of the injection pen into the pen cap.

Embodiment 11. The method of any one of embodiments 1-10, wherein receiving information from one or more detectors of the pen cap of the medicine delivery device comprises receiving information from an injection pen insertion level detector.

Embodiment 12. The method of embodiment 11, wherein receiving information from the injection pen insertion level detector comprises receiving information regarding a proximity of an end of an injection pen to an end of the pen cap opposite an opening of the pen cap.

Embodiment 13. The method of any one of embodiments 1-12, wherein receiving information from one or more detectors of the pen cap of the medicine delivery device comprises receiving information from an injection pen pull force detector.

Embodiment 14. The method of embodiment 13, wherein receiving information from the injection pen pull force detector comprises receiving information regarding an amount of force being experienced by an injection pen in direction parallel to a longitudinal axis of the pen cap.

Embodiment 15. The method of any one of embodiments 1-14, wherein receiving information from one or more detectors of the pen cap of the medicine delivery device comprises receiving information from a clasping force detector.

Embodiment 16. The method of embodiment 15, wherein receiving information from the clasping force detector comprises receiving information regarding an amount of force being exerted on an injection pen by the pen cap.

Embodiment 17. A medicine delivery device comprising: an injection pen; and a pen cap comprising: one or more detectors; a vibration motor; and a controller comprising: at least one processor; and at least one non-transitory computer-readable storage medium storing instructions thereon that, when executed by the at least one processor, cause the controller to: receive information from the one or more detectors of the pen cap regarding physical interactions between the pen cap and the injection pen; analyze the received information to identify at least one of an incorrect physical interaction or a correct physical interaction between the pen cap and the injection pen; and responsive to identifying the at least one of an incorrect physical interaction or a correct physical interaction between the pen cap and the injection pen, provide feedback via the vibration motor of the medicine delivery device.

Embodiment 18. The medicine delivery device of embodiment 17, wherein the one or more detectors of the pen cap comprise one or more of an injection pen entry detector, an injection pen insertion level detector, an injection pen pull force detector, or a clasping force detector.

Embodiment 19. The medicine delivery device of any one of embodiments 17 or 18, further comprising instructions that, when executed by the at least one processor, cause the controller to: identify at least one of an incorrect dosing action or a correct dosing action; and responsive to identifying the at least one of an incorrect dosing action or a correct dosing action, provide feedback via the vibration motor of the medicine delivery device.

Embodiment 20. A non-transitory computer-readable medium storing instructions thereon that, when executed by at least one processor, cause the at least one processor to perform steps comprising: receiving information regarding physical interactions between a pen cap and an injection pen of a medicine delivery device; analyzing the received information to identify at least one incorrect physical interaction between the pen cap and the injection pen; and responsive to identifying the at least one incorrect physical interaction, providing haptic feedback via the pen cap.

The embodiments of the disclosure described above and illustrated in the accompanying drawing figures do not limit the scope of the invention, since these embodiments are merely examples of embodiments of the invention, which is defined by the appended claims and their legal equivalents. Any equivalent embodiments are intended to be within the scope of this invention. Indeed, various modifications of the present disclosure, in addition to those shown and described herein, such as alternative useful combinations of the content features described, may become apparent to those skilled in the art from the description. Such modifications and embodiments are also intended to fall within the scope of the appended claims and legal equivalents.

What is claimed is:

1. A method of providing feedback to a user of a medicine delivery device, comprising:
receiving information from a detector of a pen cap of the medicine delivery device;
analyzing the received information to identify at least one of incorrect usage of the medicine delivery device or correct usage of the medicine deliver device; and
responsive to identifying the at least one of incorrect usage or correct usage, providing feedback via at least one feedback component of the pen cap of the medicine delivery device.

2. The method of claim 1, wherein identifying at least one of incorrect usage or correct usage comprises identifying physical interactions between the pen cap and an injection pen of the medicine delivery device.

3. The method of claim 1, wherein identifying at least one of incorrect usage or correct usage comprises identifying a capping event between the pen cap of the medicine delivery device and an injection pen of the medicine delivery device.

4. The method of claim 1 wherein identifying at least one of incorrect usage or correct usage comprises identifying an uncapping event between the pen cap of the medicine delivery device and an injection pen of the medicine delivery device.

5. The method of claim 1, wherein identifying at least one of incorrect usage or correct usage comprises identifying insertion of a new injection pen into the pen cap of the medicine delivery device.

6. The method of claim 1, wherein identifying at least one of incorrect usage or correct usage comprises identifying an incomplete insertion of an injection pen of the medicine delivery device into the pen cap of the medicine delivery device.

7. The method of claim 1, wherein identifying at least one of incorrect usage or correct usage comprises identifying a complete insertion of an injection pen of the medicine delivery device into the pen cap of the medicine delivery device.

8. The method of claim 1, wherein identifying at least one of incorrect usage or correct usage comprises identifying insertion of an injection pen of the medicine delivery device with at least one of a needle or a sheath into the pen cap of the medicine delivery device.

9. The method of claim 1, wherein receiving information from a detector of the pen cap of the medicine delivery device comprises receiving information from an injection pen entry detector.

10. The method of claim 9, wherein receiving information from the injection pen entry detector comprises receiving information regarding entry of an injection pen into the pen cap or removal of the injection pen into the pen cap.

11. The method of claim 1, wherein receiving information from a detector of the pen cap of the medicine delivery device comprises receiving information from an injection pen insertion level detector.

12. The method of claim 11, wherein receiving information from the injection pen insertion level detector comprises receiving information regarding a proximity of an end of an injection pen to an end of the pen cap opposite an opening of the pen cap.

13. The method of claim 1, wherein receiving information from a detector of the pen cap of the medicine delivery device comprises receiving information from an injection pen pull force detector.

14. The method of claim 13, wherein receiving information from the injection pen pull force detector comprises receiving information regarding an amount of force being experienced by an injection pen in direction parallel to a longitudinal axis of the pen cap.

15. The method of claim 1, wherein receiving information from a detector of the pen cap of the medicine delivery device comprises receiving information from a clasping force detector.

16. The method of claim 15, wherein receiving information from the clasping force detector comprises receiving information regarding an amount of force being exerted on an injection pen by the pen cap.

17. A medicine delivery device comprising:
an injection pen; and
a pen cap comprising:
one or more detectors;
a vibration motor; and
a controller comprising:
at least one processor; and
at least one non-transitory computer-readable storage medium storing instructions thereon that, when executed by the at least one processor, cause the controller to:
receive information from the one or more detectors of the pen cap regarding physical interactions between the pen cap and the injection pen;
analyze the received information to identify at least one of an incorrect physical interaction or a correct physical interaction between the pen cap and the injection pen; and
responsive to identifying the at least one of an incorrect physical interaction or a correct physical interaction between the pen cap and the injection pen, provide feedback via the vibration motor of the medicine delivery device.

18. The medicine delivery device of claim 17, wherein the one or more detectors of the pen cap comprise one or more of an injection pen entry detector, an injection pen insertion level detector, an injection pen pull force detector, or a clasping force detector.

19. The medicine delivery device of claim 17, further comprising instructions that, when executed by the at least one processor, cause the controller to:
identify at least one of an incorrect dosing action or a correct dosing action; and
responsive to identifying the at least one of an incorrect dosing action or a correct dosing action, provide feedback via the vibration motor of the medicine delivery device.

20. A non-transitory computer-readable medium storing instructions thereon that, when executed by at least one processor, cause the at least one processor to perform steps comprising:
receiving information regarding physical interactions between a pen cap and an injection pen of a medicine delivery device;
analyzing the received information to identify at least one incorrect physical interaction between the pen cap and the injection pen; and
responsive to identifying the at least one incorrect physical interaction, providing haptic feedback via the pen cap.
